(19) United States
(12) Patent Application Publication
(10) Pub. No.: US 2019/0269478 A1
(43) Pub. Date: Sep. 5, 2019

(54) FIXING STAND

(71) Applicants: KYUSHU UNIVERSITY, NATIONAL UNIVERSITY CORPORATION, Fukuoka-shi, Fukuoka (JP); HOGY MEDICAL CO., LTD., Tokyo (JP)

(72) Inventors: Ryu NAKADATE, Fukuoka-shi (JP); Makoto HASHIZUME, Fukuoka-shi (JP); Shunsuke NAGAI, Tokyo (JP); Hiroyasu FUJITA, Tokyo (JP); Jiro KATO, Tokyo (JP)

(73) Assignees: KYUSHU UNIVERSITY, NATIONAL UNIVERSITY CORPORATION, Fukuoka-shi, Fukuoka (JP); HOGY MEDICAL CO., LTD., Tokyo (JP)

(21) Appl. No.: 16/344,949

(22) PCT Filed: Aug. 25, 2017
(86) PCT No.: PCT/JP2017/030531

§ 371 Date: Apr. 25, 2019

(30) Foreign Application Priority Data
Oct. 26, 2016 (JP) 2016-210031

(51) Int. Cl. A61B 90/50; A61B 1/00; G02B 23/24; A61B 50/24

(52) U.S. Cl. CPC A61B 90/50; A61B 50/24; G02B 23/2476; A61B 1/00147

(57) ABSTRACT

A fixing stand to which an endoscope body and treatment instruments can be easily attached in an easy-to-operate manner and which facilitates increase or decrease in the number of such items is provided.

A fixing stand includes legs and a fixing stand body erected on the legs, the fixing stand allowing at least one or more of an endoscope body of an endoscope and an operation input portion of a treatment instrument for use in a surgery to be attached to the fixing stand in a detachable manner. The endoscope body and the operation input portion are attached to fixtures which are disposed side by side substantially horizontally to the direction in which the fixing stand is erected. The fixtures are detachably attached to a fixing base which extends substantially horizontally to the fixing stand body. A hanger component is further attached to the fixing base.

### TECHNICAL FIELD

The present invention relates to a fixing stand for holding and fixing medical appliances used in a surgery, such as endoscopes and treatment instruments.

### BACKGROUND ART

A laparoscopic surgery with an endoscope is typically performed by two persons: an endoscope operator who operates an endoscope for observing the interior of the abdominal cavity and a surgeon who conducts the surgery while viewing an image of the abdominal cavity captured by the endoscope. During the surgery, the endoscope operator continues holding the endoscope and also performs operations such as changing the orientation of the endoscope or advancing and retreating it in the insertion direction via oral instructions or the like when the surgeon wants to change the observation location at the operative site or the angle of view during the surgery. For this type of surgery method, communication between the surgeon and the endoscope operator is very important. Due to issues associated with space in an operation room and/or the necessity for smooth communication as just mentioned, there is a demand for the ability to hold and fix of an endoscope so that the surgeon can also operate the endoscope.

Various methods are known for such fixing of an endoscope. For example, an endoscope holder having an arm for holding an endoscope, a supporting portion on which an endoscope holder is fixed, and multiple joints is known, such as one described in Patent Literature 1. The arm, the supporting portion and the joints are respectively equipped with handles, and the arm, the supporting portion and the joints are fixed and released by operating the handles so that the endoscope is held in a movable or fixable manner.

More recently, approaches like endoscopic submucosal dissection (ESD) have been also put into practice. The ESD involves inserting a treatment instrument through the mouth, the anus and the like and removing a piece of surface layer of mucosa over a wide area of the stomach or the large intestine without piercing through the wall of the stomach or the large intestine. Further, an approach called Natural Orifice Translumenal Endoscopic Surgery (NOTES) is known. The NOTES involves inserting a flexible endoscope such as a stomach or intestine camera through the mouth, the anus, the vagina or the urethra, which are naturally present in the body surface, further delivering the flexible endoscope to the abdominal cavity piercing through the wall of the stomach or the large intestine, and performing diagnosis or treatment of an organ in the abdominal cavity.

Such translumenal endoscopic surgery represented by the endoscopic submucosal dissection (ESD) performs treatment and the like by inserting treatment instruments such as forceps and a knife along with a flexible endoscope through the mouth or some other opening naturally present in the body surface, and delivering them to the site of disease. Thus, it can reduce invasion to the human body by causing no damage to the body surface and eliminating complication such as infection or adhesion of the abdominal wall, which can occur in a traditional surgery.

A treatment instrument for use in such a translumenal endoscopic surgery has a bending portion for bendably operating the treatment instrument as inserted in a flexible endoscope and projecting from a tip of the flexible endoscope, as described in Patent Literature 2. The treatment instrument also has a sheath wire portion for transmitting bending motions to the bending portion, and an operation input portion for operating the bending motion of the bending portion by pushing and pulling of the sheath wire.

### CITATION LIST

### Patent Literature

Patent Literature 1: Japanese Patent Laid-Open No. 2003-325436
Patent Literature 2: Japanese Patent Laid-Open No. 2010-511440

### SUMMARY OF INVENTION

### Technical Problem

However, because the endoscope holder described in Patent Literature 1 fixes the endoscope by inserting it into a through hole formed in the arm, it has the problem of being unable to hold bending treatment instruments or the like simultaneously. With the fixing stand described in Patent Literature 2, the endoscope body and the operation input portion are directly attached to the fixing stand. Thus, the position of attachment and/or the number of items for attachment cannot be easily changed and items cannot be conveniently positioned for the surgeon.

The present invention was made to solve these problems; specifically, an object thereof is to provide a fixing stand to which an endoscope body and treatment instruments can be easily attached in an easy-to-operate manner and which facilitates increase or decrease in the number of such items.

### Solution to Problem

A fixing stand according to the present invention for attaining the object is a fixing stand including legs and a fixing stand body erected on the legs, the fixing stand allowing at least one or more of an endoscope body of an endoscope and an operation input portion of a treatment instrument to be attached to the fixing stand in a detachable manner. The endoscope body and the operation input portion are attached to fixtures which are disposed side by side substantially horizontally to a direction in which the fixing stand is erected. The fixtures are detachably attached to a fixing base which extends substantially horizontally to the fixing stand body. A hanger component is further attached to the fixing base.

In the fixing stand according to the present invention, the fixing base is preferably attached to the fixing stand body via a height adjustment mechanism.

In the fixing stand according to the present invention, the endoscope body is preferably attached to the fixing base via a joint component.

In the fixing stand according to the present invention, the joint component preferably includes a first joint component to which the endoscope body is attached and a second joint component to be attached to at least one of the fixtures, and the first joint component and the second joint component are preferably coupled to each other in a bendable manner.

In the fixing stand according to the present invention, an endoscope gripping arm for gripping an insertion portion extending from the endoscope body is preferably attached to at least one of the fixtures.

### Advantageous Effect of Invention

With the present invention, the endoscope body and the operation input portion are attached to the fixtures detachably attached to the fixing base. Thus, the positions of the endoscope body and the operation input portion can be changed as desired and the number of fixtures can be changed as well. This enables positioning of the endoscope and treatment instruments at locations convenient for the surgeon, which can improve the efficiency of a surgery.

### BRIEF DESCRIPTION OF DRAWINGS

FIG. 1 is a schematic illustration for describing how a fixing stand according to an embodiment is used.

FIG. 2 is a schematic illustration for generally describing a surgery method with bending treatment instruments.

FIG. 3 is a side view for describing the structure of a bending treatment instrument for use in an endoscopic surgery.

FIG. 4 is a perspective view of a fixing stand according to the embodiment.

FIG. 5 shows the fixing device with an endoscope attached.

FIG. 6 is a perspective view of the fixing device.

FIG. 7 is a perspective view of a holder body of the fixing device.

FIG. 8 is an exploded view of the holder body of the fixing device.

### DESCRIPTION OF EMBODIMENT

A fixing stand according to the present invention is described below with reference to the drawings. The embodiment described below is not intended to limit the subject matters set forth in the claims and not all of the combinations of features described in the embodiment are essential for the solution of the invention.

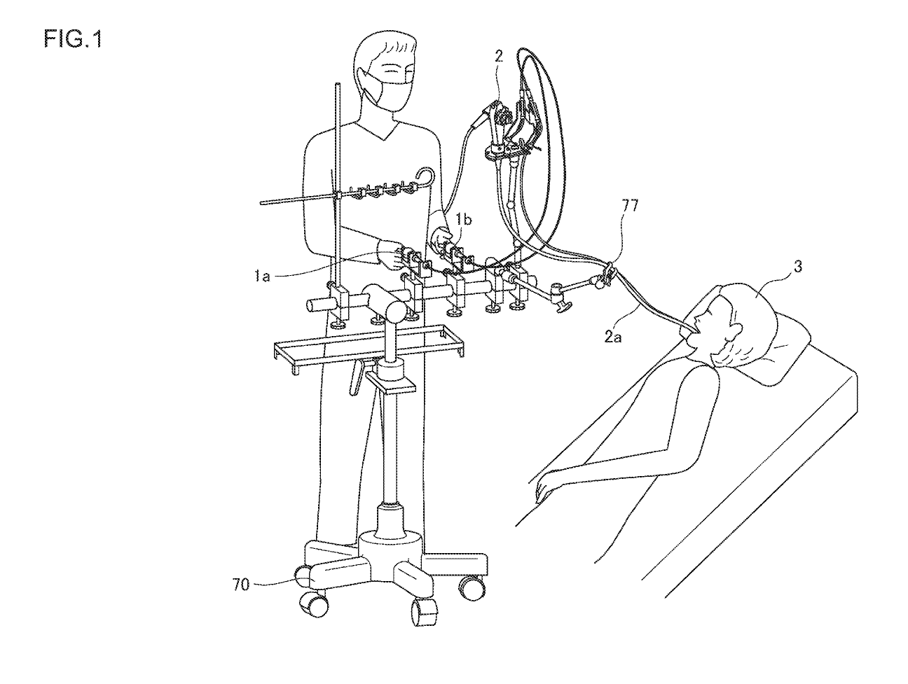

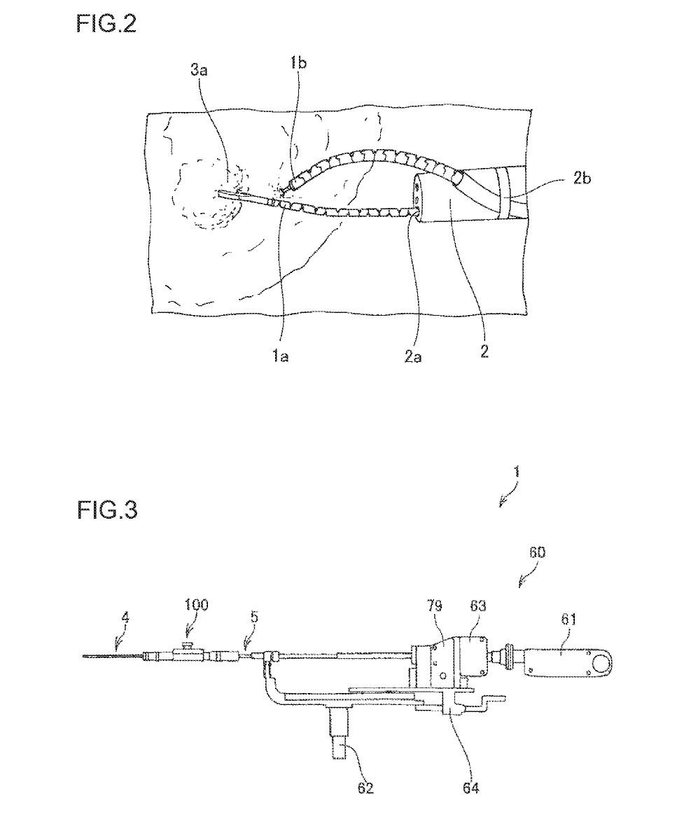

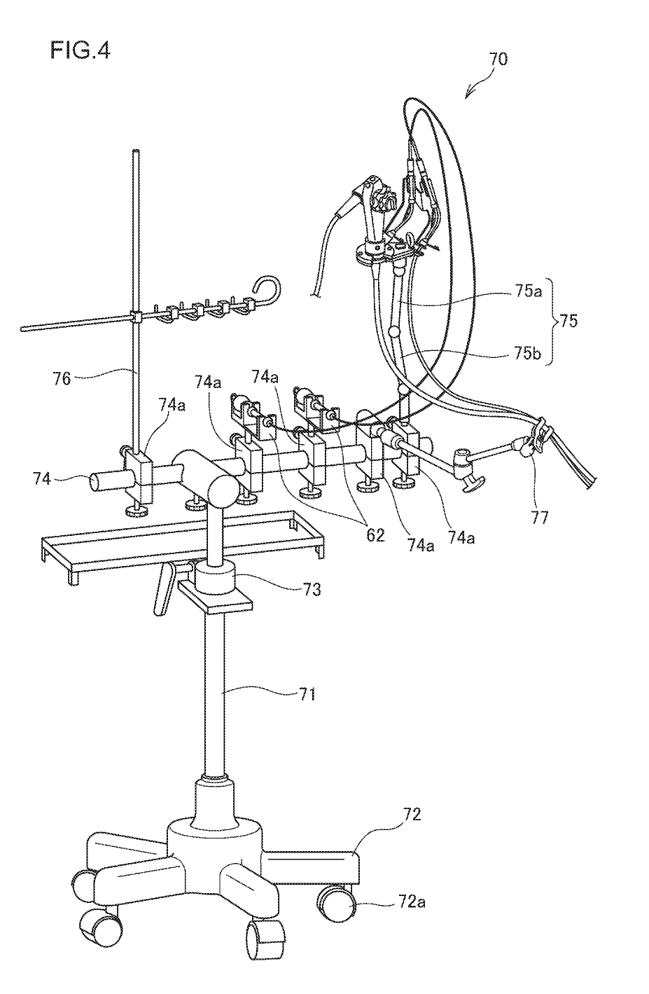

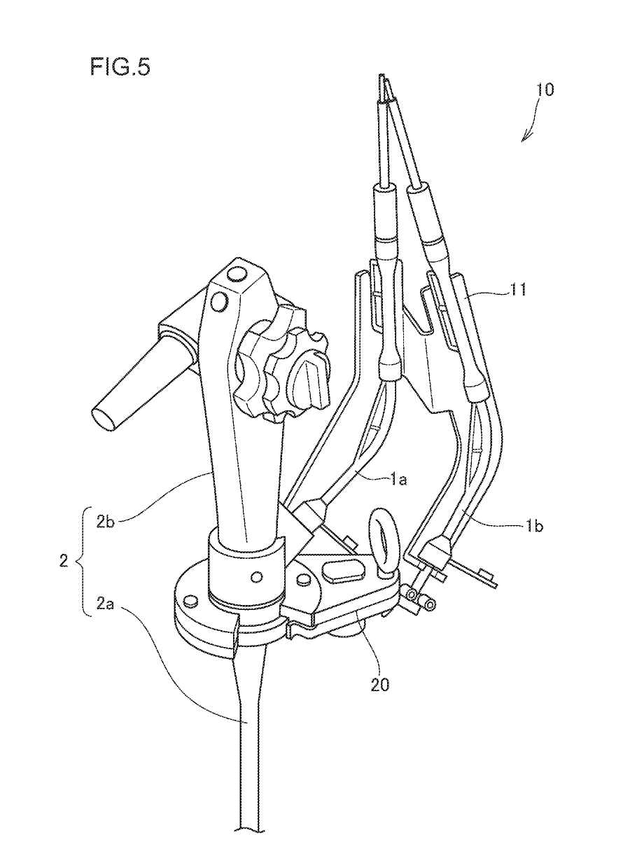

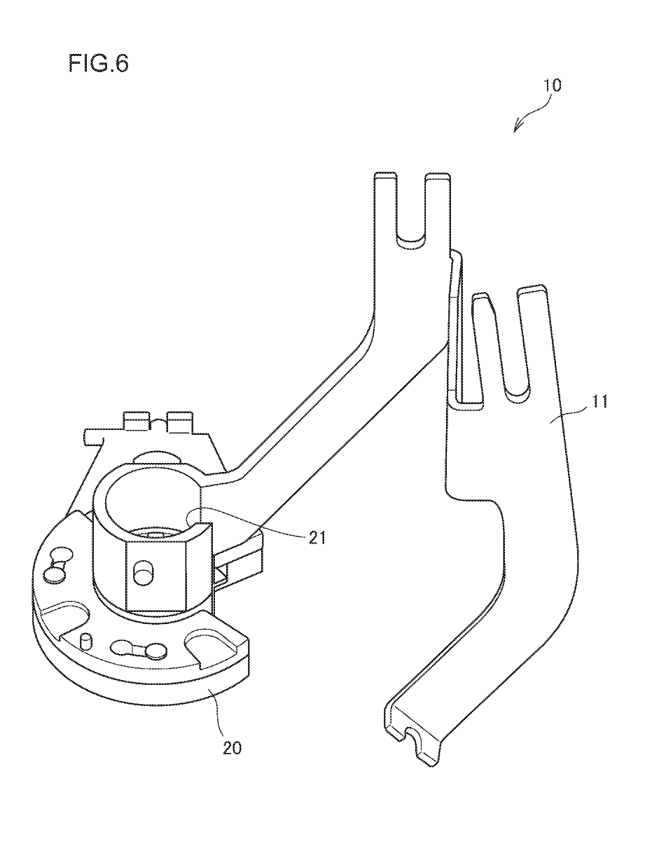

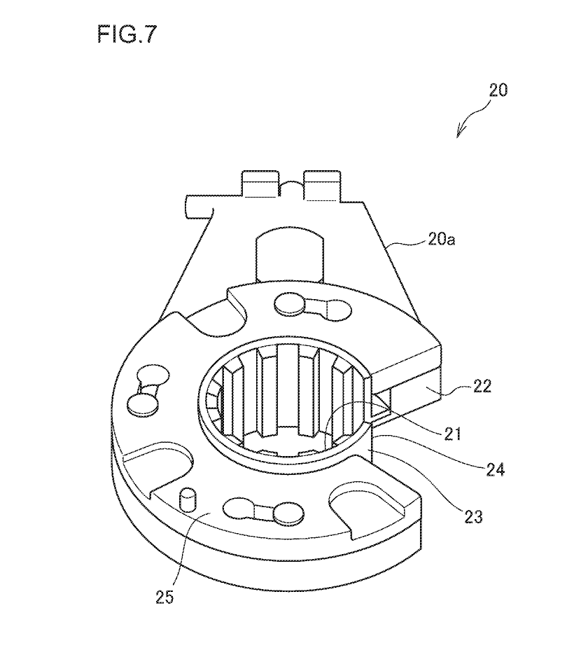

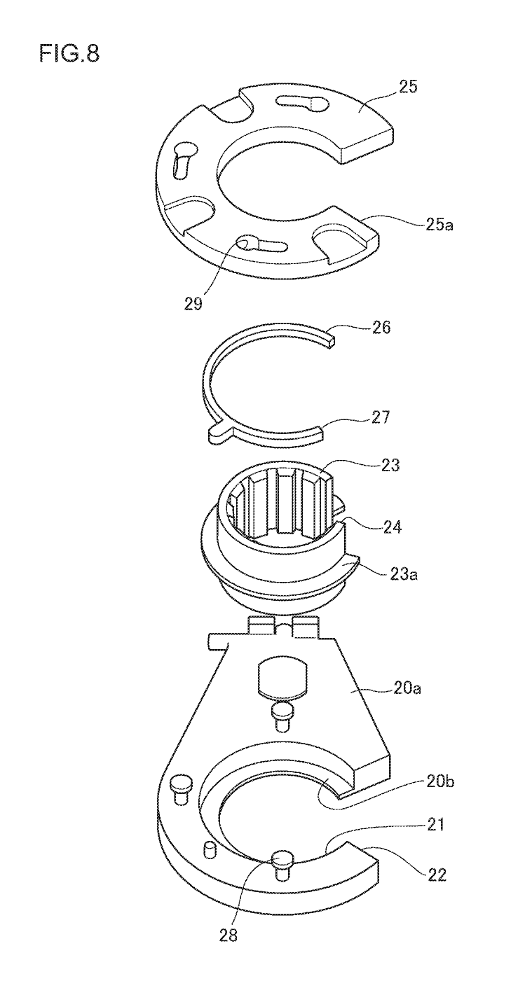

FIG. 1 is a schematic illustration for describing how a fixing stand according to an embodiment is used; FIG. 2 is a schematic illustration for generally describing a surgery method with bending treatment instruments; FIG. 3 is a side view for describing the structure of a bending treatment instrument for use in an endoscopic surgery; FIG. 4 is a perspective view of a fixing stand according to the embodiment; FIG. 5 shows the fixing device with an endoscope attached; FIG. 6 is a perspective view of the fixing device; FIG. 7 is a perspective view of a holder body of the fixing device; and FIG. 8 is an exploded view of the holder body of the fixing device.

As shown in FIGS. 1 and 2, in a laparoscopic surgery with an endoscope, an insertion portion 2a of a flexible endoscope 2 is inserted into a patient 3 through his/her mouth, anus or the like. Then, diagnosis is made on an affected area 3a, such as a cancer in an abdominal cavity like a digestive organ, using images of the interior of the abdominal cavity captured by the flexible endoscope 2. Alternatively, the affected area 3a is removed using a bending treatment instrument 1a inserted into an endoscope channel and a bending treatment instrument 1b which is attached to the insertion portion 2a by a treatment instrument insertion tube 2b attached to the insertion portion 2a and is inserted into the patient from his/her mouth, anus or the like along with the insertion portion 2a. For the bending treatment instruments 1a and 1b, a bending treatment instrument for forceps 1a equipped with forceps at its tip and a bending treatment instrument for knife 1b equipped with an electric knife at its tip are advantageously used.

The bending treatment instrument for forceps 1a and the bending treatment instrument for knife 1b bend individually and independently from the flexible endoscope 2 so as to have at least two-degree-of-freedom. It is thus possible to grip or exsect the affected area 3a while fixing the point of view of the flexible endoscope 2, enabling a procedure with a stable field of view and high degree of freedom. As noted above, the bending treatment instrument for forceps 1a and the bending treatment instrument for knife 1b are different in the component attached to their tips. Thus, they will be referred to as bending treatment instrument 1 in the following description; the bending treatment instrument for forceps 1a and the bending treatment instrument for knife 1b are to be encompassed by the bending treatment instrument 1.

As shown in FIG. 3, the bending treatment instrument 1 includes forceps which are attached to the tip of a bending portion 4 having two-degree-of-freedom in the horizontal and vertical directions and are fitted in an openable and closeable manner by a device wire as discussed below, or an electric knife (not shown). The bending treatment instrument 1 also includes an operation input portion 60 for making bending motions of the bending portion 4, opening and closing motions of the forceps, and advancing and retracting of the electric knife, and a sheath wire portion 5 including multiple wires for transmitting the operation of the operation input portion 60 and a sheath into which the wires are inserted.

The operation input portion 60 is attached on a fixing stand connector 62 via a direct acting device 64, which is capable of sliding an operation input portion body 63 in the longitudinal direction. The operation input portion 60 allows the forceps, the bending portion 4, and the sheath wire portion 5 to be pushed and pulled along the longitudinal direction by sliding the direct acting device 64 in the longitudinal direction, thereby adjusting the amount of projection of the forceps from the endoscope channel 2c or from the treatment instrument insertion tube 2b. The fixing stand connector 62 is attached on a fixing stand 70. As the bending treatment instrument 1 is a well-known treatment instrument, it is not described in detail herein.

As shown in FIG. 4, a fixing device 10 is used by being attached to the fixing stand 70 according to this embodiment. The fixing stand 70 includes a fixing stand body 71, multiple legs 72 attached to one end of the fixing stand body 71, and a fixing base 74 attached at the other end of the fixing stand body 71 via a height adjustment mechanism 73. Each of the legs 72 has a caster 72a attached thereon, being configured to facilitate the movement of the fixing stand 70. Preferably, the casters 72a are configured to permit locking and unlocking of rolling. Such a configuration can prevent an unintended movement of the fixing stand 70 while the fixing stand 70 is in use.

The fixing base 74 is a rod-shaped component extending from the fixing stand body 71 substantially in the horizontal direction. A joint component 75, the fixing stand connector 62, a hanger component 76, and an endoscope gripping arm 77 are each attached on the fixing base 74 via a fixture 74a.

The joint component 75 includes a first joint component 75a and a second joint component 75b, which are configured to be bendable so that the position of an endoscope body 2b attached to the fixing device 10 can be fixed at a desired position. The joint component may include two or more joint components if they can be configured to be bendable relative to one another. In this embodiment, the fixing device 10 is attached to one end of the first joint component 75a and a fixture 74a is attached to one end of the second joint component 75b, with their other ends being coupled with each other in a bendable manner.

The hanger component 76 is a component on which treatment instruments and the like used in the surgery are temporarily kept. By keeping sterilized treatment instruments and the like on it, they can be prevented from making contact with unclean objects such as other treatment instruments and the like, so that the sterilized state of the treatment instruments being kept on the hanger component can be maintained. For holding treatment instruments and the like on the hanger component 76, various methods may be applied. For example, the hanger component 76 may be advantageously configured to grip treatment instruments in a clipping fashion.

The endoscope gripping arm 77 is a component for gripping the insertion portion 2a in order to prevent it from falling off due to the self-weight of the flexible endoscope 2 or the insertion portion 2a even in a case where the flexible endoscope 2 is fixed to the fixing stand 70 by gripping the insertion portion 2a when the insertion portion 2a of the flexible endoscope 2 is inserted into the patient 3 through his/her mouth, anus and the like, as shown in FIG. 1. Since the insertion portion 2a is thus gripped by the endoscope gripping arm 77, a surgery can be performed by a single surgeon without requiring an endoscope operator by fixing the flexible endoscope 2 to the fixing stand 70. In this embodiment, the endoscope gripping arm 77 is described for a case where it has a clip structure and the insertion portion 2a is gripped by the clip structure. However, the tip structure of the endoscope gripping arm 77 is not limited to a clip structure and may be any of various structures that can appropriately fix the insertion portion 2a.

As shown in FIG. 5, the fixing device 10 is configured such that the endoscope body 2b of the flexible endoscope 2 can be fixed to the holder body 20. Specifically, a through hole into which the endoscope body 2b can be inserted is formed in the holder body 20, and the endoscope body 2b is held by inserting the endoscope body 2b into the through hole.

The fixing device 10 also has a horn component 11 attached on the holder body 20. By fixing the shaft fixing portions of the bending treatment instruments 1a and 1b to the horn component 11, the movement ranges of the bending treatment instruments 1a and 1b are limited so as not to hinder persons or objects when the flexible endoscope 2 is turned in order to handle the flexible endoscope 2.

As shown in FIG. 6, the fixing device 10 is provided with a detaching mechanism, which allows the endoscope body 2b to be detached from the holder body 20 while keeping the endoscope body 2b attached in the through hole 21 of the holder body 20 and the insertion portion 2a inserted in a body cavity.

Referring to FIGS. 7 and 8, the structures of the detaching mechanism and the holder body 20 are described. As shown in FIG. 7, the holder body 20 includes a holder body base 20a with the through hole 21 having a first cutout 22 formed therein, a turning component 23 attached in the through hole 21, a pressing device 26 for pressing the turning component 23 in the axial direction of the through hole 21, and a lid component 25 for fixing the turning component 23 and the pressing device 26 in the axial direction relative to the holder body base 20a.

At the through hole 21 of the holder body base 20a, a retainer portion 20b to engage with a flange 23a of the turning component 23 is formed. The turning component 23 is a substantially C-shaped tubular component with a second cutout 24 corresponding to the first cutout 22, and has the flange 23a extending in the radial direction formed on its outer peripheral surface. Since the turning component 23 turns by sliding against the holder body base 20a, it is preferably made of synthetic resin with heat resistance and high rigidity in order to deter abrasion caused by sliding and/or prevent creation of dust.

Also, the turning component 23 is pressed by the pressing device 26 in the axial direction, and the pressing device 26 has a third cutout 27 formed therein corresponding to the first cutout and the second cutout 24. The pressing device 26 may be any component that can press the flange 23a of the turning component 23 in the axial direction. For example, a wave washer or the like is advantageously used as an adjustment spring.

Further, the turning component 23 and the pressing device 26 are fixed in the axial direction by the lid component 25 attached to the holder body base 20a. The lid component 25 has a fourth cutout 25a corresponding to the first cutout 22, the second cutout 24 and the third cutout 27, and also has an engagement groove 29 formed therein. The engagement groove 29 is configured to be mate with an engaging protrusion 28 which is formed on the holder body base 20a so as to correspond to the engagement groove 29, so that the lid component 25 can be fixed to the holder body base 20a. In this case, the engagement groove 29 has an insertion part formed to be slightly larger than the diameter of the head of the engaging protrusion 28 and a fixing part formed to be smaller than the diameter of the head of the engaging protrusion 28. By then inserting the engaging protrusion 28 into the insertion part of the engagement groove 29 and rotating the lid component 25 in the circumferential direction, the engaging protrusion 28 is moved into the fixing part of the engagement groove 29, thereby fixing the holder body base 20a and the lid component 25 together.

Being thus configured, the fixing device 10 allows the turning motion of the endoscope body 2b fitted in the turning component 23 because the turning component 23 is fitted in the through hole 21 of the holder body 20. Additionally, since the turning component 23 is pressed in the axial direction by the pressing device 26, reasonable frictional resistance is given when the turning component 23 is turned, preventing the turning component 23 from turning under load as low as the self-weight of the endoscope body 2b. This enables holding of the endoscope body 2b without having a specific locking mechanism for the turning component 23.

Also, due to the formation of the first to fourth cutouts 22, 24, 27, 25a in the holder body 20, the turning component 23, the pressing device 26 and the lid component 25, it is easy to detach the endoscope body 2b from the holder body 20 by aligning the positions of the first to fourth cutouts 22, 24, 27, 25a even while the insertion portion 2a of the flexible endoscope 2 inserted in a body cavity.

As described above, the fixing device 10 is capable of fixing the flexible endoscope 2 and/or the bending treatment instruments 1a, 1b to the fixing stand 70 together. Thus, it allows a surgeon to perform a surgery on his own using bending treatment instruments without requiring an endoscope operator to hold the flexible endoscope. It also makes it possible to immediately remove the endoscope body 2b from the fixing stand 70 where necessary, such as in an emergency, and switch to a traditional, hand-holding type surgery with the endoscope operator.

While a preferred embodiment of the present invention has been described, the technical scope of the present invention is not limited to the above-described embodiment. Various modifications or improvement may be made to the embodiment.

The fixing stand 70 according to this embodiment was described for a case where the casters 72a are attached to the legs 72. However, the legs 72 may be configured for direct placement on the floor without the attachment of the casters 72a. Such a configuration can prevent dislocation of the fixing stand 70 caused by an unintended rolling of the casters 72a.

It will be apparent from the description in the claims that forms with such modifications or improvements can fall within the technical scope of the present invention.

### REFERENCE SIGNS LIST

1a bending treatment instrument for forceps
1b bending treatment instrument for knife
2 flexible endoscope
2a insertion portion
2b endoscope body
2c endoscope channel
3 patient
3a affected area
4 bending portion
5 sheath wire portion
10 fixing device
11 horn component
20 holder body
20a holder body base
21 through hole
22 first cutout
23 turning component
23a flange
24 second cutout
25 lid component
25a fourth cutout
26 pressing device
27 third cutout
28 engaging protrusion
29 engagement groove
70 fixing stand
71 fixing stand body
72 leg
73 height adjustment mechanism
74 fixing base
75 joint component
76 hanger component

1. A fixing stand comprising legs and a fixing stand body erected on the legs, the fixing stand allowing at least one or more of an endoscope body of an endoscope and an operation input portion of a treatment instrument to be attached to the fixing stand in a detachable manner,
wherein the endoscope body and the operation input portion are attached to fixtures which are disposed side by side substantially horizontally to a direction in which the fixing stand is erected,
the fixtures are detachably attached to a fixing base which extends substantially horizontally to the fixing stand body, and
a hanger component is further attached to the fixing base.

2. The fixing stand according to claim 1, wherein the fixing base is attached to the fixing stand body via a height adjustment mechanism.

3. The fixing stand according to claim 1, wherein the endoscope body is attached to the fixing base via a joint component.

4. The fixing stand according to claim 3, wherein
the joint component comprises a first joint component to which the endoscope body is attached and a second joint component to be attached to at least one of the fixtures, and
the first joint component and the second joint component are coupled to each other in a bendable manner.

5. The fixing stand according to claim 1, wherein an endoscope gripping arm for gripping an insertion portion extending from the endoscope body is attached to at least one of the fixtures.
